(19) United States
(12) Patent Application Publication
(10) Pub. No.: US 2025/0049745 A1
(43) Pub. Date: Feb. 13, 2025

(54) COMPOSITION COMPRISING SHORT AND MEDIUM-CHAIN FATTY ACIDS AND ITS USE IN THE PROPHYLAXIS AND TREATMENT OF INFECTIONS

(71) Applicant: NUTRITION SCIENCES N.V., Drongen (BE)

(72) Inventor: Geert BRUGGEMAN, Drongen (BE)

(21) Appl. No.: 18/721,141

(22) PCT Filed: Dec. 22, 2022
(86) PCT No.: PCT/EP2022/087588

§ 371 Date: Jun. 17, 2024

(30) Foreign Application Priority Data
Dec. 23, 2021 (BE) 2021/6055

(51) Int. Cl. A61K 31/20; A61K 9/00; A61K 31/19; A61K 31/22; A61K 31/23

(52) U.S. Cl. CPC A61K 31/20; A61K 9/0056; A61K 31/19; A61K 31/22; A61K 31/23

(57) ABSTRACT

The current invention relates to compositions comprising a short and medium-chain fatty acids (SCFA-MCFAs) fraction to be used in food or feed applications.

### FIELD OF THE INVENTION

The present invention relates to the use of a specific range within the short and medium-chain fatty acids (SCFA-MCFA) and its use as an inhibitor of microbial and viral contamination and growth. In particular, the invention relates to the use of caproic acid (C6) or the salts or derivatives or mixtures or emulsions thereof, supplemented with butyric acid (C4), valeric acid (C5), heptaoic acid (C7), caprylic acid (C8), pelargonic acid (C9) or their salts or derivatives or mixtures or emulsions thereof to inhibit bacterial and viral.

### BACKGROUND

There is a need for measures that can reduce high feed costs and decrease maintenance costs of food-producing animals. This will also directly lead to the optimization of the profits. Feed additives can offer to be important tools. These can help in the fight against pathogenic infections and can effect a positive influence on, for example, feed conversion.

The positive characteristics of medium-chain fatty acids as a feed additive composition for animals have previously been known. EP 1 294 37 and EP 1 765 318 both describe a feed, supplemented with a feed additive consisting of medium-chain fatty acids having 6 to 10 carbon atoms. These additives have a positive influence on the reduction or elimination of microbial pathogens in the gastrointestinal tract of animals.

However, the current state of the art describes primarily a composition with only one or a few medium-chain chain-fatty acids. In addition, these compositions often, if they comprise multiple fatty acids, offer non-optimal ratios and are not well balanced. Good balancing of the composition of feed additives on the basis of different medium-chain fatty acids is, however, crucial. After all, a non-optimal or sub-optimal concentration of the fatty acids may have the result that it is less or even not effective when used in a feed additive composition.

There is an urgent need to develop novel compositions of short- and medium-chain fatty acids that display optimal and broad applicability, especially for animal health. Animal feed can be subjected to various types of infection. Fungi are the principal cause of spoilage of animal feed, particularly cereals and legumes. In addition, some fungal species may elaborate toxins that cause serious disease in animals consuming the feed. Bacterial spoilage may also occur. For instance, in the United States, animal feed is frequently contaminated with non-Typhi serotypes of Salmonella enterica and may lead to infection or colonization of food animals. Common bacteria associated with feed infections comprise E. Coli, Salmonella, Streptococcus Sp., Lactobacillus Sp., Clostridia, listeria and Enterobacteria. Globally, fungal and/or bacterial infections can reduce animal health, decrease meat and derivative production, increase costs for housing and nutrition, and impact human health.

Viruses are another source of infection. In recent years, outbreaks of several viruses have challenged the agricultural industry such as the swine industry worldwide. Common viruses associated with feed infections comprise Foot and Mouth Disease (FMD), Newcastle disease, Avian influenza, Porcine epidemic diarrhoea (PED) and African swine fever (ASF).

It is known that feed may carry pathogens that are detrimental to animal health and welfare. As such, the spreading of microbial or viral particles via the feed is an example of growing concern. To this purpose, antiseptic products such as formaldehyde are commonly used to disinfect the feed. But these products can in turn exert negative effects.

U.S. Pat. No. 10,695,393 describes a composition for feed that can include a saponin composition including saponins and at least one medium-chain fatty acid (MCFA) or ester or salt thereof. The saponins and at least one MCFA present in the composition can work synergistically to prevent methanogenesis by bacteria.

US 2021 0 046 144 describes methods of inhibiting the spread of African swine fever virus and/or classical swine fever virus in animal feed, feed ingredients, and pet food. U.S. Pat. No. 10,918,118 describes methods of preventing or decreasing PEDv and/or Salmonella bacteria in animal feed, feed ingredients, and pet food.

EP 3 457 865 describes an animal feed additive as effective as a broad-spectrum preservative, said feed additive comprises as active ingredients propionic acid, glyceryl propionates, hexanoic acid (C6) and optionally minor amounts of an alkali metal salt and/or a C7-C12 fatty acid.

WO 2009 072 097 discloses an antimicrobial composition comprising a blend of two or more free fatty acids.

The use of short- and medium-chain fatty acids against oral microorganisms is also described by Chifu al., 2011 (Short- and medium-chain fatty acids exhibit antimicrobial activity for oral microorganisms).

Peh et al., 2020 (Antimicrobial activity of organic acids against Campylobacter spp. and development of combinations. A synergistic effect?) disclose testing the activity of organic acids, including fatty acids C1, C2, C3, C6 and C8, alone or in combinations against Campylobacter jejuni and Campylobacter coli.

Additional problems are presented by the source of fatty acids used as feed supplements in animals. The use of environmentally challenging vegetable oils (such as palm oil) for the production of short- and medium-chain fatty acids will lead to detrimental effects in the longer term. There is a need to replace these with more ecologic alternatives.

The present invention aims to resolve at least some of the problems and disadvantages mentioned above.

### SUMMARY OF THE INVENTION

The present invention and embodiments thereof serve to provide a solution to one or more of the above-mentioned disadvantages. To this end, the present invention relates to composition suited for use as a feed additive comprising a short and medium-chain fatty acids (SCFA-MCFAS) fraction wherein said SCF-MCFA fraction comprises fatty acids selected from C2 to C12 or esters or salts or mixture thereof. Preferred embodiments of the compositions are discussed in claims 1 to 22.

In a further aspect, the present invention also relates to a composition according to any of the claims 23 to 26, for use as a feed additive for use in the treatment, suppression and/or prevention of microbial and/or viral infections in animals.

In a final aspect, the present invention relates to a method of inhibiting microbial and/or viral infections in animal feed or drinking water, said method comprising dosing a composition according to any of the claims 27 to 30 to the animal feed or drinking water.

### DETAILED DESCRIPTION OF THE INVENTION

There is a need for new compositions that can reduce high feed costs and decrease maintenance costs of food-producing animals. This will also directly lead to the optimization of the profits. Feed additives can be important tools. These can help in the fight against pathogenic infections and can effect a positive influence on, for example, feed conversion. Feed compositions should be efficient and have broad-spectrum applicability. The current invention highlights a composition comprising multiple components that provide optimal efficacy.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention and form different embodiments, as would be understood by those in the art. For example, in the following claims, any of the claimed embodiments can be used in any combination.

The term “short-chain fatty acid” or “SCFA” as used herein, refers to fatty acids with a short-chain length, wherein the fatty acids may be saturated or unsaturated. In the present invention, the longest continuous chain of an SCFA can consist of 1 to 5 carbon atoms, for example, formic acid (C1), acetic acid (C2), propionic acid (C3), butyric acid (C4) or valeric acid (C5). The term SCFA also refers to short-chain fatty acids that are chemically modified, and to short-chain fatty acids that are provided with side-chains, such as, without limitation, one or more alkyl groups, preferably C1-C10 alkyl groups. As described herein, the term “short-chain fatty acid (SCFA) derivative” refers to a fatty acid chain of which the carboxyl group is reversibly converted to a different group, preferably, but without limitation, to an amide, salt, ester or glyceride such as, for example, a mono-, di- or tri-glyceride.

The term “medium-chain fatty acid” or “MCFA” as used herein, refers to fatty acids with a medium-chain length, wherein the fatty acids may be saturated or unsaturated. In the present invention, the longest continuous chain of an MCFA can consist of 6 to 12 carbon atoms, for example, caproic acid (C6), caprylic acid (C8), pelargonic acid (9), capric acid (C10) or lauric acid (C12). The term MCFA also refers to medium-chain fatty acids that are chemically modified, and to medium-chain fatty acids that are provided with side-chains, such as, without limitation, one or more alkyl groups, preferably C1-C10 alkyl groups. As described herein, the term “medium-chain fatty acid (MCFA) derivative” refers to a fatty acid chain of which the carboxyl group is reversibly converted to a different group, preferably, but without limitation, to an amide, salt, ester or glyceride such as, for example, a mono-, di- or tri-glyceride.

The term “Feed Conversion Ratio” or “FCR” describes the efficiency with which the animals convert feed into the desired output. For animals raised for meat (such as beef cows, pigs, chickens and fish) the output is the flesh, that is, the body mass gained by the animal represented either in the final mass of the animal or the mass of the dressed output.

The term “antiviral in vitro assay” as used herein refers to any method suitable to determine the potential efficacy of an antiviral substance, compound or composition towards a chosen virus or viral family. Known methods comprise TCID₅₀ assays, EC₅₀/CC₅₀ assays, plaque assays, HAI (hemagglutination inhibition) assays and ELISA/Luminex.

Cytopathic effect or cytopathogenic effect (abbreviated CPE) is to be understood as structural changes in host cells that are caused by viral invasion. The infecting virus causes lysis of the host cell or when the cell dies without lysis due to an inability to reproduce. Both of these effects occur due to CPEs. If a virus causes these morphological changes in the host cell, it is said to be cytopathogenic. Common examples of CPE include rounding of the infected cell, fusion with adjacent cells to form syncytia, and the appearance of nuclear or cytoplasmic inclusion bodies. CPEs and other changes in cell morphology are only a few of the many effects of cytocidal viruses. When a cytocidal virus infects a permissive cell, the viruses kill the host cell through changes in cell morphology, cell physiology, and the biosynthetic events that follow. These changes are necessary for efficient virus replication but at the expense of the host cell.

Without wanting to be limitative, a virus may be viruses belonging to Coronaviruses, Orthomyxoviruses, Paramyxoviridae viruses, Pneumoviruses, Rubulaviruses, Paramyxoviruses, Metapneumoviruses and Morbilliviruses, Poxviridae, Orthopoxvirus such as Variola vera, Picornaviruses, Enteroviruses, Rhinoviruses, Heparnaviruses, Cardioviruses, Aphthoviruses, Bunyavirus, Heparnaviruses, Filoviruses, Togaviruses, Flaviviruses, Pestiviruses, Hepadnaviruses, Other hepatitis viruses, Rhabdoviruses, Caliciviridae, Retroviruses, Reoviruses, Parvoviruses, Herpesviruses, Papovaviruses, Adenoviruses. Common viruses associated with feed infections comprise Foot and Mouth Disease (FMD), Newcastle disease, Avian influenza, Porcine epidemic diarrhoea (PED) and African swine fever (ASF).

Without wanting to be limitative, a fungus may be a chosen from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Without wanting to be limitative, a bacteria may be chosen from Neisseria meningitidis, Streptococcus pneumoniae, Streptococcus pyogenes, Moraxella catarrhalis, Bordetella pertussis, Staphylococcus aureus, Clostridium tetani, Corynebacterium diphtheriae, Haemophilus influenzae, Pseudomonas aeruginosa, Streptococcus agalactiae, Chlamydia trachomatis, Chlamydia pneumoniae, Helicobacter pylori, Escherichia coli, Bacillus anthracis, Yersinia pestis, Staphylococcus epidermis, Clostridium perfringens or Clostridium botulinums, Legionella pneumophila, Coxiella burnetiid, Brucella, Francisella, Neisseria gonorrhoeae, Treponema pallidum, Haemophilus ducreyi, Enterococcus faecalis or Enterococcus faecium, Staphylococcus saprophyticus, Yersinia enterocolitica, Mycobacterium tuberculosis, Rickettsia, Listeria monocytogenes, Vibrio cholerae, Salmonella typhi, Borrelia burgdorferi, Porphyromonas gingivalis, Klebsiella.

According to the current invention, SCFA-MCFAs are used to prevent and/or inhibit microbial, fungal and/or viral diseases of animals.

The term “animal” means a human or another animal likely to develop or suffer from microbial, fungal and/or viral disease, including avian, bovine, canine, equine, galline, feline, hircine, lapine, murine, musteline, ovine, piscine, porcine and vulpine animals. Preferably, the target is a human, bovine, canine, feline, galline, ovine, porcine or avian.

The present invention concerns specific compositions of SCFA and MCFA that are suitable to be used as a feed additive, as well as methods for treating, inhibiting or lowering the microbial and/or viral titer in feed, feed ingredients or drinking waters of animals.

In a first aspect, the invention relates to specific compositions suited for use as a feed additive comprising a short and medium-chain fatty acids (SCFA-MCFAS) fraction.

In a first embodiment, said composition comprises an SCFA-MCFA fraction wherein said SCF-MCFA fraction comprises fatty acids selected from C2 to C12 or esters or salts or mixture thereof. More, in particular, said fraction comprises at least 15% C6 or esters, salts or mixtures thereof, and further at least between 0.1% and 30 weight % C4; between 0.1% and 5 weight % C5; between 0.1% and 10 weight % C7; between 0.1% and 35 weight % C8; between 0.1% and 20 weight % C9; minimally 5 weight % of C2 and optionally C3 and wherein the total amount of components in said SCFA-MCFA does not exceed 100%. In a further embodiment, said SCF-MCFA fraction may comprise between 15 to 99% of C6, preferably between 20 to 95% of C6, between 25 to 90% of C6, between 30 to 85% of C6, between 35 to 80% of C6. In a further embodiment, the composition may comprise at least 5% of C2, at least 6% of C2, at least 7% of C2, at least 8% of C2, at least 9% of C2.

Propionic acid (C3) is today widely used as an effective and well-proven preservative in animal feedstuffs. However, there can be certain problems associated with the use of propionic acid. Since it is corrosive to skin and metals and has a flashpoint of less than 55° C., it is classified as corrosive and flammable, thus limiting the possibility for end-users to store and transport the material. Additionally, a higher amount of C3 will cause an unpleasant taste in the feed, leading to lower feed consumption. According to an embodiment, the amount of propionic acid (C3) is less than 5%, preferably less than 4%, preferably less than 3%, preferably less than 2%, preferably less than 1% in said SCF-MCFA fraction.

In the current invention, C2 and C3 are generated as trace elements of the biological production method of the SCFAs and MCFAS.

In another embodiment, the present invention relates to a composition comprising a short and medium-chain fatty acid (SCFA-MCFA) fraction wherein said SCF-MCFA fraction comprises fatty acids selected from C2 to C12 or esters or salts or mixture thereof, wherein said fraction comprises between 15 to 99% of C6 and/or an ester or salt thereof, and further C4, C5, C7, C8 and C9, or esters, salts or a mixture thereof, wherein the total amount of C4, C5, C6, C7, C8, C9 or esters, salts or a mixture thereof amounts to at least 0.5 weight % and maximal 100% of said SCFA-MCFA fraction.

In an embodiment, the minimal amount of C4 to C9 in said fraction is 0.5, 1, 2, 3, 4, 5, 6, 7, 8, 9, 10, 15, 20, 21, 22, 23, 24, 25, 26, 27, 28, 29, 30 weight %.

In an embodiment, the maximal amount of C4 to C9 in said fraction is 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65 weight %.

In an embodiment, the total amount of C4, C5, C6, C7, C8, C9 or esters, salts or a mixture thereof in said SCFA-MCFA fraction is between 0.5 and 100, 1 and 100, 2 and 100, 3 and 100, 4 and 100, 5 and 100, 6 and 100, 7 and 100, 8 and 100, 9 and 100, 10 and 100, 11 and 100, 12 and 100, 13 and 100, 14 and 100, 15 and 100, 16 and 100, 17 and 100, 18 and 100, 19 and 100, 20 and 100, 21 and 100, 22 and 100, 23 and 100, 24 and 100, 25 and 100, 26 and 100, 27 and 100, 28 and 100, 29 and 100, 30 and 100, 35 and 100, 40 and 100, 45 and 100, 50 and 100, 55 and 100, 60 and 100, 65 and 100, 70 and 100, 75 and 100, 80 and 100, 85 and 100, 90 and 100, 95 and 100 or 99 and 100 weight %.

In another embodiment, the total amount of C4, C5, C6, C7, C8, C9 or esters, salts or a mixture thereof in said SCFA-MCFA fraction is between 0.5 and 99, 0.5 and 98, 0.5 and 98, 0.5 and 97, 0.5 and 96, 0.5 and 95, 0.5 and 94, 0.5 and 93, 0.5 and 92, 0.5 and 91, 0.5 and 90, 0.5 and 89, 0.5 and 88, 0.5 and 87, 0.5 and 86, 0.5 and 85, 0.5 and 84, 0.5 and 83, 0.5 and 82, 0.5 and 81, 0.5 and 80, 0.5 and 79, 0.5 and 78, 0.5 and 77, 0.5 and 76, 0.5 and 75, 0.5 and 74, 0.5 and 73, 0.5 and 72, 0.5 and 71, 0.5 and 70, 0.5 and 65, 0.5 and 60, 0.5 and 55, 0.5 and 50, 0.5 and 45, 0.5 and 40, 0.5 and 35, 0.5 and 30, 0.5 and 25, 0.5 and 20, 0.5 and 15, 0.5 and 10, 0.5 and 5 or 0.5 and 1 weight %.

The inventors have further found that a composition comprising a mixture of SCFA-MCFAs or derivatives as described above shows optimal preventive and curative properties towards microbial and/or viral disease. In an embodiment, the SCF-MCFA fraction may comprise at least 15% C6 or esters, salts or mixtures thereof, and further at least
between 0.1 and 30 weight % C4, more preferably between 0.5 and 15 weight % C4;
between 0.1 and 5 weight % C5, more preferably between 0.5 and 2.5 weight % C5;
between 0.1 and 10 weight % C7, more preferably between 0.5 and 5 weight % C7;
between 0.1 and 35 weight % C8, more preferably between 0.5 and 17.5 weight C8;
between 0.1 and 20 weight % C9, more preferably between 0.5 and 10 weight % C9; and
minimally 5 weight % of C2 and/or maximally 5 weight % and optionally C3.

In a further embodiment, said SCF-MCFA fraction may comprise between 15 to 99% of C6, preferably between 20 to 95% of C6, between 25 to 90% of C6, between 30 to 85% of C6, between 35 to 80% of C6. In a further embodiment, said SCF-MCFA fraction may comprise at least 5% of C2, at least 6% of C2, at least 7% of C2, at least 8% of C2, at least 9% of C2. According to an embodiment, the amount of propionic acid (C3) is less than 10%, preferably less than 5%, preferably less than 4%, preferably less than 3%, preferably less than 2%, preferably less than 1%.

In yet another embodiment, the present invention relates to a composition suited for use as a feed additive, comprising a short and medium-chain fatty acids (SCFA-MCFAs) fraction wherein said SCF-MCFA fraction comprises fatty acids selected from C2 to C12 or esters or salts or mixture thereof, wherein said SCFA-MCFA fraction comprises at least 15% of a C6 component, wherein said C6 component is a mixture of a C6 salt and/or ester and a C6 acid and wherein the concentration of C6 in acid form is higher than said C6 in salt and/or ester form. More preferably at least 50% of the total amount of C6 is present in said acid form, more preferably at least 55%, 60%, 65%, 70%, 75% to 80%.

It was unexpectedly found by the inventors that while having a mixture of acids and esters/salts in the SCFA-MCFA fraction is important, better results were obtained by compositions in which the amount of acids in said fraction was higher.

In a further embodiment the SCF-MCFA fraction of the composition may comprise at least 15% C6 or esters, salts or mixtures thereof, and further
at least between 0.1 and 30 weight % C4; more preferably between 0.5 and 15 weight % C4;
between 0.1 and 5 weight % C5, more preferably between 0.5 and 2.5 weight % C5;
between 0.1 and 10 weight % C7, more preferably between 0.5 and 5 weight % C7;
between 0.1 and 35 weight % C8, more preferably between 0.5 and 17.5 weight C8;
between 0.1 and 20 weight % C9, more preferably between 0.5 and 10 weight % C9; and minimally 5 weight % of C2 and/or maximally 5 weight % and optionally C3.

In a further embodiment, said SCF-MCFA fraction of the composition may comprise between 15 to 99% of C6, preferably between 20 to 95% of C6, between 25 to 90% of C6, between 30 to 85% of C6, between 35 to 80% of C6. In a further embodiment, the said fraction may comprise at least 5% of C2, at least 6% of C2, at least 7% of C2, at least 8% of C2, at least 9% of C2. According to an embodiment, the amount of propionic acid (C3) is less than 10%, preferably less than 5%, preferably less than 4%, preferably less than 3%, preferably less than 2%, preferably less than 1%.

In another embodiment, the present invention relates to a composition suited for use as a feed additive, comprising a short and medium-chain fatty acids (SCFA-MCFAs) fraction wherein said SCF-MCFA fraction comprises fatty acids selected from C2 to C12 or esters or salts or mixture thereof, wherein said SCFA-MCFA fraction comprises at least 15% of C6 or esters, salts or mixtures thereof and further C4, C5, C7, C8 and C9, or esters, salts or a mixture thereof and wherein the total amount of SCFA-MCFAs in acid form in said SCFA-MCFA fraction is higher than said the total amount of SCFA-MCFA in salt and/or ester form. More preferably at least 50% of the total amount of SCFA-MCFA is present in said acid form, more preferably at least 60%, more preferably at least 70% even more preferably at least 80%.

In an embodiment, the SCF-MCFA fraction of the composition may comprise at least 15% C6 or esters, salts or mixtures thereof, and further at least between 0.1 and 30 weight % C4; between 0.1 and 5 weight % C5; between 0.1 and 10 weight % C7; between 0.1 and 35 weight % C8; between 0.1 and 20 weight % C9; minimally 5 weight % of C2 and/or maximally 5 weight % and optionally C3. In a further embodiment, said fraction may comprise between 15 to 99% of C6, preferably between 20 to 95% of C6, between 25 to 90% of C6, between 30 to 85% of C6, between 35 to 80% of C6. In a further embodiment, the fraction may comprise at least 5% of C2, at least 6% of C2, at least 7% of C2, at least 8% of C2, at least 9% of C2. According to an embodiment, the amount of propionic acid (C3) is less than 10%, preferably less than 5%, preferably less than 4%, preferably less than 3%, preferably less than 2%, preferably less than 1%. The use of SCFA-MCFA salt and/or esters reduces the unpleasant smell associated with the acid form. Moreover, the solubility of SCFA-MCFA salt and/or esters is increased in comparison to the acid form.

The inventors unexpectedly found that a minimum of 15 weight % of caproic acid (C6) in the compositions according to the invention increased the efficiency. The inventors have further found that said compositions comprising a mixture of SCFA-MCFAs or derivatives as described above shows optimal preventive and curative properties towards microbial and/or viral disease, whether it is through its preventive or disinfecting nature, or whether it is due to treatment.

According to the current invention, short- and medium-chain fatty acids are generated through more ecologic alternatives. These are generated amongst others, by enzymatic carbon chain elongation, oxidizing unsaturated fatty acids, oxidative cleavage. Non-limiting examples include US 2020 0 325 095 and WO 2009 083 174. US 2020 0 325 095 uses catalysts based on copper oxide which has lower intrinsic toxicity than the catalysts commonly used industrially. These catalysts are also in a form that enables them to be more easily separated from the reaction environment, reducing the possibilities of contamination of the product in comparison with the homogeneous catalysts commonly used. WO 2009 083 174 describes an enzymatic production of a fatty alcohol having 6-18 carbon atoms and/or a fatty acid comprising 8-18 carbon atoms, by carbon chain elongation, involving organic compounds comprising 2-6 carbon atoms; subjecting the organic compounds to enzymatic carbon chain elongation in the presence of an electron donor and separating the obtained fatty alcohol and/or fatty acid. The new resulting product is better soluble in water compared to classic SCFA-MCFAs, as they constitute mono- or dicarboxylic acids, salts or esters. Additionally, these compounds do not have the distinct odor associated with the acid forms.

In an embodiment, said SCF-MCFA fraction of said compositions according to the invention comprise at least 40% caproic acid (C6). In the state of the art, compositions suited for use as a feed additive either comprise mainly longer chain MCFAs or an equal ratio of MCFAs. However, the inventors found that a minimum of 40 weight % of caproic acid is beneficial and shows optimal preventive and curative properties towards microbial and/or viral disease. By preference, said SCF-MCFA fraction of said composition comprises at least 41 weight %, at least 42 weight %, at least 43 weight %, at least 44 weight %, at least 45 weight %, at least 46 weight %, at least 47 weight %, at least 48 weight %, at least 49 weight %, at least 50 weight %, at least 51 weight %, at least 52 weight %, at least 53 weight %, at least 54 weight %, at least 55 weight %, at least 56 weight %, at least 57 weight %, at least 58 weight %, at least 59 weight %, at least 60 weight % caproic acid (C6).

In an embodiment, said SCF-MCFA fraction of said compositions according to the invention comprise at least 70% caproic acid (C6). In the state of the art, compositions suited for use as a feed additive either comprise mainly longer chain MCFAs or an equal ratio of MCFAs. However, the inventors found that a minimum of 70 weight % of caproic acid is beneficial and shows optimal preventive and curative properties towards microbial and/or viral disease. By preference, said SCF-MCFA fraction of said composition comprises at least 71 weight %, at least 72 weight %, at least 73 weight %, at least 74 weight %, at least 75 weight %, at least 76 weight %, at least 77 weight %, at least 78 weight %, at least 79 weight % caproic acid (C6), at least 80, at least 85, at least 90 weight % caproic acid (C6).

In a further preferred embodiment, said SCF-MCFA fraction of said compositions comprise at least 10% acetic acid (C2). In a further embodiment, the composition may comprise at least 11% of C2, at least 12% of C2, at least 13% of C2, at least 14% of C2, at least 15% of C2, at least 16% of C2, at least 17% of C2, at least 18% of C2, at least 19% of C2, at least 20% of C2.

By preference, said SCF-MCFA fraction of said compositions according to the current invention comprise maximally 10% propionic acid (C3). In a further embodiment, said SCF-MCFA fraction of said composition may comprise maximally 5% C3, preferably maximally 4% C3, preferably maximally 3% C3, preferably maximally 2% C3, preferably maximally 1% C3.

In an embodiment, said SCF-MCFA fraction of the compositions according to the current invention comprise between 20 to 95%, preferably between 25 to 90% of C6 and/or an ester or salt thereof, and further C4, C5, C7, C8 and C9, or esters, salts or a mixture thereof. In a preferred embodiment said SCF-MCFA fraction of said composition comprises between 30 to 85%, preferably between 35 to 80% of C6 and/or an ester or salt thereof, and further C4, C5, C7, C8 and C9, or esters, salts or a mixture thereof.

In an embodiment, said SCF-MCFA fraction of said compositions according to the invention comprise both the acid form of SCFA-MCFAs and the salt/ester form of SCFA-MCFAs. It has been reported that the acid forms of SCFA-MCFAs have a very distinctive and often unpleasant odor while salts or esters do not exhibit this feature. Moreover, the water solubility of SCFA-MCFA acids is lower compared to the salt and/or ester form. However, acid forms were reported to be more active against pathogens than their salt and/or ester forms. The inventors, therefore, discovered that the mix of the different SCFA-MCFA forms as disclosed in the current embodiments is beneficial. It allows animals to better feed, reduces aversion to the feed composition and it aids the digestion and uptake of the fatty acids.

In an embodiment, the composition of the invention has a higher total amount of SCFA-MCFAs in acid form in said SCFA-MCFA fraction than said the total amount of SCFA-MCFA in salt and/or ester form. Preferably, at least 50% of the total amount of SCFA-MCFA is present in said acid form more preferably at least 55%, at least 60%, at least 65%, at least 70%, at least 75%, even more preferably at least 80%. In a particularly preferred embodiment, the SCFA-MCFA fraction in said compositions will comprise between 70 and 80% of SCFA-MCFAs in free acid form and between 20 and 30% of SCFA-MCFA in salt and/or ester form.

Depending on the target animal species, the ratio between the acid and salt/ester form can vary. Preferably the ratio between SCFA-MCFA in the acid form and SCFA-MCFA in the salt/ester form is between 10:1 and 2:1, preferably the ratio between SCFA-MCFA in the acid form and SCFA-MCFA in the salt/ester form is between 8:1 and 2:1, preferably the ratio between SCFA-MCFA in the acid form and SCFA-MCFA in the salt/ester form is between 6:1 and 2:1, preferably the ratio between SCFA-MCFA in the acid form and SCFA-MCFA in the salt/ester form is between 5:1 and 2:1 such as 4:1.

In an embodiment, the C6 component of the composition is a mixture of a C6 salt and/or ester and a C6 acid and the concentration of C6 in acid form is higher than said C6 in salt and/or ester form. Preferably, at least 50% of the total amount of C6 is present in said acid form, more preferably at least 60%, more preferably at least 70% even more preferably at least 80%. Depending on the target animal species, the ratio between the acid and salt and/or ester form can vary. Preferably the ratio between SCFA-MCFA in the acid form and SCFA-MCFA in the salt and/or ester form is between 10:1 and 2:1, preferably the ratio between SCFA-MCFA in the acid form and SCFA-MCFA in the salt and/or ester form is between 8:1 and 2:1, preferably the ratio between SCFA-MCFA in the acid form and SCFA-MCFA in the salt and/or ester form is between 6:1 and 2:1, preferably the ratio between SCFA-MCFA in the acid form and SCFA-MCFA in the salt and/or ester form is between 5:1 and: 1, such as 4:1.

In an embodiment, the total amount of the short and medium-chain fatty acids (SCFA-MCFAs) fraction in the composition is comprised between 1% and 100% based on the total weight or volume of the composition. Preferably, the concentration of the short- and medium-chain fatty acids, as described herein, amounts at least 1, 5, 10, 15, 20, 25, 30, 35, 40, 45, 50, 55, 60, 65, 70, 75, 80, 85, 90, 95, 99% by weight or volume of the composition/feed additive. In a preferred embodiment, the total amount of the short and medium-chain fatty acids (SCFA-MCFAs) fraction in the composition is at least 20%, at least 50% based on the total weight or volume of the composition. In a further embodiment, the short- and medium-chain fatty acids, as described herein, amounts to (based on dry weight) between 1 g/100 g composition (1% by weight) and 100 g/100 g composition (100% by weight), preferably between 50 g/100 g and 99 g/100 g (50-99% by weight), 50 g/100 g and 95 g/100 g (50-95% by weight), more preferably 50 g/100 g and 90 g/100 g composition (50-90% by weight), more preferably between 60 g/100 g and 80 g/100 g. It is to be understood that the concentration of the SCFA-MCFAs such as described herein maximally amounts to 100% by weight of the composition. The inventors have found that within the ranges mentioned above, the efficiency of the composition/feed additive for use in the prevention, treatment and/or suppression of microbial and/or viral diseases is further increased.

According to a further or another embodiment, the compositions according to the current invention may further comprise one or more organic acids, preferably chosen from the group of lactic acid, formic acid, citric acid, oxalic acid, malic acid, or combinations thereof. The organic acids comprised herein result in a composition that is soluble in water and therefore is ideally suited for oral administration by admixing the composition with animal drinking water.

The compositions according to some embodiments may further comprise one or more stabilizers and/or emulsifiers which improve the mixability of the composition in animal drinking water. Such stabilizers may comprise glycerol.

In further embodiments, the compositions may further comprise vitamins, minerals, trace elements or a combination thereof. Vitamins include but are not limited to Vitamin A, Vitamin D3, Vitamin E, Vitamin K3, Thiamin, Riboflavin, Panthothenic acid, Biotin, Folic acid, Vitamin B12, Niacin, Pyridoxine, Ascorbic acid, Inositol and Choline. Minerals and trace elements include but are not limited to magnesium, sodium, manganese, iron, zinc, copper, selenium, phosphorous, cobalt and iodine. The addition of these components further contributes to a complete and healthy diet of the animals and positively influences their zootechnical performance. The inclusion of these components in the composition simplifies the steps needed for feeding the animals as these components no longer need to be added separately to the feed, resulting in improved efficiency of the feeding process. In addition, the provision of SCFA-MCFAs and/or SCFA-MCFA derivatives in the animals' feed was found to result in an increase in feed palatability, which in turn leads to animals that eat more, grow bigger, faster and/or healthier, which eventually leads to an increase in the profitability of the swine production.

In further embodiments, the compositions according to the invention as described herein is formulated as a liquid or as a solid form. The term “solid form” means a powder, a granule or a pellet in particular. The term “liquid form”, in particular, means a solution in water or means a solution in oil and includes a viscous paste and a non-viscous solution. The SCFA-MCFAs and/or SCFA-MCFA derivatives as described above are oil-soluble and can be provided both as powder and as an oil solution. In particular, the composition is suitable for oral administration. A feed comprising the composition according to an embodiment of the invention is produced or manufactured by means known to a person skilled in the art. In an embodiment, the feed comprising the composition according to the invention is provided as a dry extruded feed pellet. This formulation allows a relatively long shelf life and also permits the packaging and storage of large amounts of feed.

In an embodiment, the composition is administered to a human subject or an animal, including avian, bovine, canine, equine, galline, feline, hircine, lapine, murine, musteline, ovine, piscine, porcine and vulpine animals. Preferably, said animal bovine, canine, feline, galline, ovine, porcine or avian. The composition is administered to a feed or drinking water, preferably as a powder, a granule, a pellet, a liquid or a paste.

The compositions of the invention can be conveniently used in the treatment, suppression and/or prevention of microbial and/or viral infections, as it can be taken orally or administered orally to animals. Therapeutically effective amounts of the composition are administered orally to said animal, preferably by adding said composition to the feed or drinking water of said animal.

Oral administration to animals of the composition is attained by feeding the composition to the animals. This method of administration provides the advantage that it is easy to perform and it does not impose the burden of large investments for novel equipment.

Administration to humans can be achieved by providing the composition according to the current invention as a tablet, a powder, preferably a water-soluble powder, a gel, a liquid.

Oral administration of the composition can be performed by admixing the animal feed with the composition of the invention prior to feeding, by feeding the animal with an animal feed already comprising the composition. Accordingly, the invention provides a feed or feed ingredient comprising a feed additive/composition for use as described above, wherein the dosage of said composition in said feed or feed ingredient is between 25 ppm and 10000 ppm by weight, between 100 ppm and 10000 ppm by weight, between 100 ppm and 9000 ppm by weight, between 500 and 8000 ppm by weight, more preferably between 1000 and 7000 ppm by weight, more preferably between 1000 and 6000 ppm by weight, even more preferably between 1000 and 5000 ppm by weight, such as 1250 ppm, 1500 ppm, 2000 ppm or 3750 ppm based on the total weight of said feed.

Alternatively, said dosage of the SCFA-MCFA fraction in said feed, feed ingredient or drinking water is between 100 ppm and 10000 ppm by weight, between 500 ppm and 9000 ppm by weight, between 1000 and 8000 ppm by weight, more preferably between 1000 and 7000 ppm by weight, more preferably between 1000 and 6000 ppm by weight, even more preferably between 1000 and 5000 ppm by weight, such as 1250 ppm, 1500 ppm, 2000 ppm or 3750 ppm.

According to a further or another embodiment, the dosages as herein described result in treatment of animals with a daily dosage of between 25 and 3000 ppm, preferably between 100 and 2500 ppm, more preferably between 200 and 2000 ppm of SCFA-MCFAs.

Alternatively, the inclusion ratio of the composition in feed is between 0.01 and 1%, preferably between 0.05 and 1%, preferably between 0.1 and 1%, preferably between 0.5 and 1%. In another embodiment the inclusion ratio of the composition in feed is between 0.01 and 1%, between 0.01 and 0.5%, between 0.01 and 0.1%, between 0.01 and 0.05%.

In a further embodiment, the feed further comprises other well-known ingredients so as to provide a nutritionally balanced complete food, including, but not limited to, plant matter, e.g., flour, meal, starch or cracked or processed grain produced from a crop plant such as wheat or other cereals, alfalfa, corn, oats, potato, rice, soybeans or other legumes; cellulose in a form that may be obtained from wood pulp, grasses, plant leaves, and waste plant matter such as rice or soybean hulls, or corn cobs; animal matter, e.g., fish or crustacean meal, oil, protein or solubles and extracts, krill, meat meal, bone meal, feather meal, blood meal, or cracklings; algal matter; yeast; bacteria; vitamins, minerals, and amino acids; organic binders or adhesives; and chelating agents and preservatives.

Preferably, the animals are treated at least once per day, more preferably two or more times per day such as, for example, 2-6 or 4-6 times per day. It is preferred that any excess food for treatment by oral administration be removed after the feeding period, e.g., by flushing out of a raceway system, or through removal out of the feed trunks.

A further aspect of the present invention relates to a method of inhibiting microbial and/or viral infections in a food product, animal feed, feed ingredient or drinking water, said method comprises dosing a composition according to any of the embodiments as described above to said animal feed, feed ingredient or drinking water. The method in accordance with the present invention uses an effective amount of a chemical mitigant to inhibit microbial and/or viral infections, in animal feed or drinking water, for example, to concentrations below the levels of detection through cytopathic effect (CPE), RT-PCR and/or virus isolation in cell culture. As used herein, an “effective amount” refers to an amount capable of providing bioavailable levels of the active compound (e.g., short/medium-chain fatty acids) sufficient to achieve the desired performance improvement.

In an embodiment, the method of lowering the microbial and/or viral titer in a food product, an animal feed, feed ingredient or drinking water, comprises adding an effective dose of said composition to said food product, animal feed, feed ingredient or drinking water. According to a further embodiment, said composition is added at a concentration of between 25 and 10.000 ppm to said food product, feed, feed ingredients or drinking water.

In an embodiment, the present invention relates to a method of enhancing the zootechnical performance of animals and/or the feed conversion ratio, said method comprises adding an effective dose of a composition according to the present invention and as defined in any of the above embodiments.

By nature, agricultural raw materials are loaded with numerous microbic organisms. Either before or during harvesting, processing at the factory (operators, processing floor included) or during shipment, storage. Agricultural raw materials are also potential reservoirs for cross-contamination. The rapid development of bale bacteria but also pathogenic species like salmonella, E. coli and toxin-producing fungi, can lead to huge economic impact and potential dangers for the animals. According to the current invention, the addition of the SCFA-MCFAs to the feed and/or water neutralizes pathogens before they come into contact with the animals. SCFA-MCFAS are antimicrobial agents that can disrupt the phospholipid membrane surrounding membrane-enclosed pathogens such as bacteria and lipid bilayer-enveloped viruses. In terms of antibacterial activity, the compounds can inhibit bacterial growth (“bacteriostatic”) or induce bacterial cell lysis and cell killing (“bactericidal”). They can also disrupt a wide range of lipid bilayer-enveloped viruses by damaging and/or effectively destroying enveloped virus particles and compromising infectivity. SCFA-MCFAs principally exhibit antiviral activity by lysing enveloped virus particles (“virucidal”).

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present invention encompasses a variety of combinations and/or integrations of the specific embodiments described herein.

The invention is further described by the following non-limiting examples which further illustrate the invention, and are not intended to, nor should they be interpreted to, limit the scope of the invention.

### EXAMPLES

### Example 1: Influence of SCFA-MCFA on Growth and Survival of E. Coli

Six times three samples of 100 ml culture medium were simultaneously inoculated with an overnight culture of E. coli F4 (Gram-negative pathogen in the gastrointestinal tract) and further incubated at 37° C. The optical density was measured at 600 nm (OD600 nm, proportional to the amount of microbial cells present). Once an OD600 nm between 0.2 and 0.5 was achieved, the following ratios of MCFAs were added to a 1% dose in the feed:
(1) nothing was added to the first sample,
(2) 10% C6+90% (C2+C3+C4+C5+C7+C8+C9) was added to the second sample,
(3) 15% C6+85% (C2+C3+C4+C5+C7+C8+C9) was added to the third sample,
(4) 20% C6+80% (C2+C3+C4+C5+C7+C8+C9) was added to the fourth sample,
(5) 50% C6+50% (C2+C3+C4+C5+C7+C8+C9) was added to the fifth sample,
(6) 100% C6 was added to the sixth sample,

The samples were further incubated at 37° C. for 4 hours at a pH of 4.0. Incubation was put to a halt after 4 hours. The OD600 nm was measured at time 0 h and after 4 h. The results of this measurement are shown in Table 1.

TABLE 1
Antimicrobial effect of various compositions with
changing C6 contribution with adjusted residual
SCFA-MCFAs(C2 + C3 + C4 + C5 +
C7 + C8 + C9) ratios as compared to pathogenic E. Coli bacteria.

| | Fractional |
|---|---|
| | reduction |
| Contribution C6 versus remaining SCFA-MCFAs | E. coli F4 (%)* |
| negative control | 0-1 |
| 10% C6 + 90%(C2 + C3 + C4 + C5 + C7 + C8 + C9) | 23 |
| 15% C6 + 85%(C2 + C3 + C4 + C5 + C7 + C8 + C9) | 91 |
| 20% C6 + 80%(C2 + C3 + C4 + C5 + C7 + C8 + C9) | 93 |
| 50% C6 + 50%(C2 + C3 + C4 + C5 + C7 + C8 + C9) | 100 |

From Table 1 can be clearly deduced that the anti-bacterial efficacy of compositions (C6)/(C2+C3+C4+C5+C7+C8+C9) is significantly improved at concentrations of C6 equal or higher than 15%.

### Example 2: Influence of SCFA-MCFA on Growth and Survival of Aspergillus niger

Five times three samples of 100 ml culture medium were simultaneously inoculated with an overnight culture of Aspergillus niger (fungus) and further incubated at 37° C. The optical density was measured at 600 nm (OD600 nm, proportional to the amount of microbial cells present). Once an OD600 nm between 0.2 and 0.5 was achieved, the following ratios of MCFAs were added to a 1% dose in the feed:
1) nothing was added to the first sample,
(2) 10% C6+90% (C2+C3+C4+C5+C7+C8+C9) was added to the second sample,
(3) 15% C6+85% (C2+C3+C4+C5+C7+C8+C9) was added to the third sample,
(4) 20% C6+80% (C2+C3+C4+C5+C7+C8+C9) was added to the fourth sample,
(5) 100% C6 was added to the fifth sample,

The samples were further incubated at 37° C. for 4 hours at a pH of 4.0. Incubation was put to a halt after 4 hours. The OD600 nm was measured at time 0 h and after 4 h. The results of this measurement are shown in Table 2.

TABLE 2
Antifungal effect of various compositions with changing
C6 contribution with adjusted residual SCFA-MCFAs
(C2 + C3 + C4 + C5 + C7 +
C8 + C9) ratios as compared to pathogenic Aspergillus niger fungus.

| | Fractional |
|---|---|
| | reduction |
| | Aspergillus |
| Contribution C6 versus remaining SCFA-MCFAs | niger (%)* |
| negative control | 0-1 |
| 10% C6 + 90%(C2 + C3 + C4 + C5 + C7 + C8 + C9) | 34 |
| 15% C6 + 85%(C2 + C3 + C4 + C5 + C7 + C8 + C9) | 96 |
| 20% C6 + 80%(C2 + C3 + C4 + C5 + C7 + C8 + C9) | 95 |

From Table 2 can be clearly deduced that the anti-fungal efficacy of compositions (C6)/(C2+C3+C4+C5+C7+C8+C9) is significantly improved at concentrations of C6 equal to or higher than 15%.

### Example 3: Influence of C6, C2 and C4+C5+C7+C8+C9 on Growth and Survival of E coli

The samples were processed and the antimicrobial activity of the compositions was determined as described in Example 1. Several SCFA-MCFA combinations were tested. The concentrations of C6 tested were at 10%, 15%, 30% and 85% and the concentrations of C2 tested were at 0.5%, 2.5%, 5% and 10%. In half of the samples, a blend of C4+C5+C7+C8+C9 was added towards a 100% mass balance of MCFAs, wherein each MCFAs of said blend was added in equimolar concentration. The C6 used had an acid/ester ratio of 60/40.

TABLE 3
Relative kill-off of E coli bacterial of various compositions
with changing C6 and C2 contribution and in the presence or absence
of adjusted residual SCFA-MCFAs(C4 + C5 + C7 + C8 + C9) ratios

C2 (%)

| kill-off (%) | | | 0.5 | 2.5 | 5 | 10 |
|---|---|---|---|---|---|---|
| with | C6 | 10 | 14 | 9 | 17 | 29 |
| C4 + C5 + C7 + | (%) | 15 | 31 | 55 | 98 | 100 |
| C8 + C9 | | 30 | 33 | 81 | 100 | 100 |
| | | 85 | 78 | 87 | 100 | 100 |
| without | C6 | 10 | 9 | 10 | 9 | 19 |
| C4 + C5 + C7 + | (%) | 15 | 18 | 37 | 78 | 80 |
| C8 + C9 | | 30 | 28 | 73 | 95 | 99 |
| | | 85 | 61 | 79 | 100 | 100 |

### Influence of C6 and C2

The results of the measurement of the relative kill-off of E coli cells are shown in Table 3. The results indicate that the addition of C2 and C6 significantly increases the effectiveness of the SCFA-MCFA composition in inhibiting E coli development in feed, in a dose-dependent and synergistic manner.

C6 became effective in inhibiting microbial growth at doses above 15%, while at 10%, C6 did not inhibit sufficiently microbial growth. Higher doses of C6, 30% and 85%, were highly effective in inhibiting E coli cell growth, up to 100% kill-off rates.

C2 became effective in inhibiting microbial growth at a dose of 5%. Lower doses of C2, 0.5% and 2.5%, were also capable of diminishing the bacterial growth but were less effective. While the high C2 doses, 5% and 10%, have both high antimicrobial efficacy, there is no significant difference between the two, so a 5% C2 dose is sufficient for the desired inhibition of E coli cell growth in feed.

### Influence of C4+C5+C7+C8+C9

The data of Table 3 also indicate that the presence of a small fraction of C4+C5+C7+C8+C9 enhances the antimicrobial effectiveness of the composition. In the absence of C4+C5+C7+C8+C9, the effectiveness of lower dosages of C2 and C6 was decreased when compared to the same dosages but in the presence of the C4+C5+C7+C8+C9 blend. Only at 85% C6 and 5% or 10% C2, did the presence of C4+C5+C7+C8+C9 not influence the antimicrobial activity of the SCFA-MCFAS blend.

### Example 4: Influence of C6 Acid/Ester Ratio on Growth and Survival of E coli

The samples were processed and the antimicrobial activity of the compositions was determined as described in Example 1. Several SCFA-MCFA combinations were tested. In all SCFA-MCFA combinations, a 15% C6 fraction was used. Various C2 concentrations and acid/ester ratios of C6 were used, as depicted in Table 4. In half of the samples, a blend of C4+C5+C7+C8+C9 was added towards a 100% mass balance of MCFAs, wherein each MCFAs of said blend was added in equimolar concentration.

TABLE 4
Relative kill-off of E coli bacterial of various
compositions with changing C6 acid/ester ratios

| | | | C2 (%) | | | |
|---|---|---|---|---|---|---|
| kill-off (%) | | | 0.5 | 2.5 | 5 | 10 |
| with | C6 | 100/0 | 15 | 17 | 16 | 31 |
| C4 + C5 + C7 + | acid/ester | 70/30 | 23 | 53 | 90 | 89 |
| C8 + C9 | ratio | 60/40 | 31 | 55 | 98 | 100 |
| | | 50/50 | 30 | 49 | 91 | 93 |
| | | 30/70 | 27 | 30 | 45 | 76 |
| | | 0/100 | 9 | 8 | 11 | 66 |
| without | C6 | 100/0 | 13 | 11 | 12 | 29 |
| C4 + C5 + C7 + | acid/ester | 70/30 | 18 | 37 | 78 | 80 |
| C8 + C9 | ratio | 60/40 | 21 | 42 | 81 | 98 |
| | | 50/50 | 25 | 38 | 79 | 88 |
| | | 30/70 | 19 | 21 | 33 | 63 |
| | | 0/100 | 10 | 11 | 9 | 39 |

The results of the measurement of the relative kill-off of E coli cells are shown in Table 4. The results indicate that the blends that contain C6 in predominately acid form over ester, namely 60/40 were significantly more effective in inhibiting E coli development in feed. These lead to up to 100% inhibition of microbial growth in feed.

The blends with C6 in a 60/40 acid/ester form were the most effective in inhibiting microbial growth at all C2 tested concentrations in the presence, as well as in the absence of C4+C5+C7+C8+C9.

Good inhibition of E coli cell growth was also obtained with C6 in a 70/30 or 50/50 acid/ester form. However, the blends comprising predominately ester or only acid or ester C6, were less effective in inhibiting microbial activity.

The results of the experiments, especially Experiments 3 and 4, are indicative of other pathogens present in the gastrointestinal tract, and the data obtained on the E coli strain can be extrapolated to the latter.

1. A composition comprising a short and medium-chain fatty acids (SCFA-MCFAs) fraction wherein said SCFA-MCFA fraction comprises fatty acids selected from C2 to C12 or esters or salts or a mixture thereof, characterized in that said SCFA-MCFA fraction comprises at least 15% C6 or esters, salts or mixtures thereof, and further at least:
between 0.1 and 30 weight % C4;
between 0.1 and 5 weight % C5;
between 0.1 and 10 weight % C7;
between 0.1 and 35 weight % C8;
between 0.1 and 20 weight % C9;
minimally 5 weight % of C2 and optionally comprises C3;
wherein the total amount of constituents in said SCFA-MCFA does not exceed 100%.

2. A composition, said composition comprises a short and medium-chain fatty acid (SCFA-MCFA) fraction wherein said SCFA-MCFA fraction comprises fatty acids selected from C2 to C12 or esters or salts or a mixture thereof, characterized in that said fraction comprises between 15 to 99% of C6 and/or an ester or salt thereof, and further C4, C5, C7, C8 and C9, or esters, salts or a mixture thereof, wherein the total amount of C4, C5, C6, C7, C8, C9 or esters, salts or a mixture thereof amounts to at least 0.5 weight % and maximal 100% of said SCFA-MCFA fraction.

3. A composition suited for use as a feed additive, comprising a short and medium-chain fatty acids (SCFA-MCFAs) fraction wherein said SCFA-MCFA fraction comprises fatty acids selected from C2 to C12 or esters or salts or a mixture thereof, characterized in that said SCFA-MCFA fraction comprises at least 15% of a C6 component, wherein said C6 component is a mixture of a C6 salt and/or ester and C6 in acid form, and wherein the concentration of C6 in acid form is higher than said C6 in salt and/or ester form.

4. (canceled)

5. The composition according to claim 1, wherein said SCFA-MCFA fraction comprises at least 40% caproic acid (C6) and/or an ester or salt thereof.

6. The composition according to claim 1, wherein said SCFA-MCFA fraction comprises at least 70% caproic acid (C6) and/or an ester or salt thereof.

7. The composition according to claim 1, wherein said SCFA-MCFA fraction comprises at least 10% acetic acid (C2).

8. The composition according to claim 1, wherein said SCFA-MCFA fraction comprises at least 15% acetic acid (C2).

9. The composition according to claim 1, wherein said SCFA-MCFA fraction comprises maximally 10% propionic acid (C3).

10. The composition according to claim 1, wherein said SCFA-MCFA fraction comprises maximally 5% propionic acid (C3).

11. The composition according to claim 1, wherein said SCFA-MCFA fraction comprises between 20 to 95% of C6 and/or an ester or salt thereof, and further C4, C5, C7, C8 and C9, or esters, salts or a mixture thereof.

12. The composition according to claim 1, wherein said SCFA-MCFA fraction comprises between 30 to 85% of C6 and/or an ester or salt thereof, and further C4, C5, C7, C8 and C9, or esters, salts or a mixture thereof.

13. The composition according to claim 1, wherein said fraction comprises both the acid form of SCFA-MCFAs and the salt/ester form of SCFA-MCFAs.

14. The composition according to claim 5, wherein the total amount of SCFA-MCFAs in acid form is higher in said SCFA-MCFA fraction than said the total amount of SCFA-MCFA in salt and/or ester form.

15. The composition according to claim 3, wherein said C6 component is a mixture of a C6 salt and/or ester and C6 in acid form and wherein and wherein the concentration of C6 in acid form is higher than said C6 in salt and/or ester form.

16. The composition according to claim 1, wherein the total amount of SCFA-MCFA in the composition is comprised between 1% and 100% based on the total weight or volume of the composition.

17. The composition according to claim 1, wherein said composition further comprises one or more organic acids chosen from the group of lactic acid, formic acid, citric acid, oxalic acid, malic acid, or combinations thereof.

18. The composition according to claim 1, wherein said composition further comprises one or more stabilizers and/or emulsifiers.

19. The composition for use according to claim 1, wherein said composition further comprises vitamins, minerals, trace elements or a combination thereof.

20. The composition according to claim 1, wherein said composition is administered to a feed or drinking water as a powder, a granule, a pellet, a liquid or a paste.

21. (canceled)

22. Feed or drinking water, characterized in that said feed or said drinking water comprises the composition according to claim 1.

23.-30. (canceled)
